(19) United States
(12) Patent Application Publication
(10) Pub. No.: US 2007/0117175 A1
(43) Pub. Date: May 24, 2007

(54) Airborne bacterial spores as an indicator of biomass in an indoor environment

(76) Inventor: Adrian Ponce, Altadena, CA (US)

Correspondence Address: Alessandro Steinfl, Esq.;c/o LADAS & PARRY, Suite 2100, 5670 Wilshire Boulevard, Los Angeles, CA, 90036-5679, US

(21) Appl. No.: 11/453,296

(22) Filed: Jun. 13, 2006

Related U.S. Application Data
(60) Provisional application No. 60/691,530, filed on Jun. 17, 2005.
(60) Provisional application No. 60/798,090, filed on May 5, 2006.

(51) Int. Cl. C12Q 1/22; C12Q 1/04; C12M 3/00

(52) U.S. Cl. 435/31; 435/34; 435/287.1

(57) ABSTRACT

Bacterial endospore concentration is an indicator of microbial contamination in an indoor environment. In accordance with the disclosed method, a measurement of the bacterial endospore concentration of an indoor environment correlates to the microbial mass in said indoor environment.

### CROSS REFERENCE TO RELATED APPLICATIONS

This application claims priority to U.S. Provisional S/N 60/691,530 for “Airborne Bacterial Spores as an Indicator of Total Biomass in an Indoor Environment” filed on Jun. 17, 2005, and U.S. Provisional S/N 60/798,090 for “Airborne Endospore Bioburden as an Indicator of Spacecraft Cleanliness” filed on May 5, 2006, both of which are incorporated herein by reference in their entirety. This application may also be related to U.S. Ser. No. 11/332,788, “Method and Apparatus for Detecting and Quantifying Bacterial Spores on a Surface filed on Jan. 12, 2006; U.S. Ser. No. 10/306,331 filed on Nov. 27, 2002 and U.S. Ser. No. 10/355,462 filed on Jan. 31, 2003, all three of which are incorporated herein by reference in their entirety.

### STATEMENT OF GOVERNMENT SUPPORT

The invention described herein was made in the performance of work under a NASA contract, and is subject to the provisions of Public Law 96-517 (35 USC 202) in which the Contractor has elected to retain title.

### BACKGROUND

1. Field

The present disclosure relates to the field of biomass detection. In particular, a method and composition are disclosed for indicating the presence of microbial biomass in an indoor environment.

2. Description of Related Art

A rapid change in microbial concentration indicates a breakdown of environmental control either by allowing conditions that foster microbial growth (e.g., rapid biofilm formation due to temperature, humidity, and nutrient availability) or by a breakdown of environmental control/treatment hardware. These failure modes need to be detected as early as possible in order to avoid detrimental effects to those persons and/or objects present in the environment.

Currently, bioburden levels are determined using culturing assays, where microorganisms are quantified in terms of colony forming units (CFU) that become visible on growth plates after a defined incubation period—usually 3-4 days. Another problematic aspect of measuring bioburden levels, is that an array of organisms can be present in a contaminated environment, and measuring each organism individually is not practical.

Therefore, what is needed is an efficiently detectable microbial species that is an indicator of microbial contamination in an indoor environment.

### SUMMARY

According to a first aspect of the present disclosure an indicator of microbial contamination in an indoor environment is provided, said indicator comprising measured bacterial endospore concentration in said indoor environment.

According to a second aspect of the present disclosure, a method is provided for using bacterial endospore concentration, comprising the steps of measuring said bacterial endospore concentration in an indoor environment; and correlating said measured bacterial endospore concentration to biomass in said indoor environment, thereby providing an indicator for said biomass.

According to a third aspect of the present disclosure, a method is provided for indicating presence of microbial contamination in an indoor environment, comprising the step of measuring bacterial endospore concentration in said indoor environment to provide an indicator of said microbial contamination.

One advantage of the present disclosure is that it obviates any dependence on culturing microbial targets. This is an obvious advantage in view of the fact that contamination of any microbial species is not desirable, and thus further culturing of a contaminating species should be avoided.

### Brief Description of the Drawings

FIG. 1 shows a Surface Air Systems (SAS) dual head impaction air sampler.

FIG. 2 shows a schematic apparatus for imaging quantifying and counting of bacterial spores (Example 3).

FIG. 3 shows a graph of data from a time course of airborne microbial concentrations.

FIG. 4 shows airborne spore transfer from culture of BS-3160.

### DETAILED DESCRIPTION

Bacterial spores can be used as indicators of microbial contamination since a significant portion of the endemic microbial communities contain spore-formers. These spore-forming bacteria release their spores into the air as a result of many environmental influences; some examples include disruption, aerosol formation or drying.

Bacterial endospores are highly resistant, thick walled structures formed by vegetative cells during a process called sporulation, and they are quite possibly the most resistant forms of life (Hindle and Hall, 1999 Analyst, 124, 1599-1604). They are highly resistant to radiation, chemical agents, desiccation, and other normally harmful environments (Nicholson, W. L. et al. 2000. Microbiol. Molecular Biol. Rev., 64, 548). Several bacterial genera are capable of producing endospores; Bacillus and Clostridium are the two most common endospore-producing genera.

Rapid detection of bacterial spores of the present disclosure is based on dipicolinic acid (DPA)-triggered lanthanide ion (terbium, europium) luminescence (Hindle and Hall, 1999 Analyst, 124, 1599-1604). The core of bacterial spores contains up to 1 molar (M) DPA that can be released into bulk solution by lysis of the spore (Murell, 1969, Bact. Spore 1, 216). The released DPA binds the lanthanide ions with high affinity and triggers intense green luminescence under UV excitation.

Spore-forming bacteria of the genus Bacillus are frequently among the most abundant genera in aerobic biofilms [Lutterbach and deFranca, 1997, Revista De Microbiologia, 28, 106-109; Singh et al., 2003, Applied and Environmental Microbiology, 69, 3412-3420; Lutterbach and deFranca, 1997, Braz. J. Chem Eng. (online), vol. 14.], and are likewise among the most abundant genera found, for example, in the air and on surfaces of the International Space Station and Mir Space Station [Pierson, 2001, Gravitational and Space Biology Bulletin, vol. 14]. As a biofilm grows, the population of the pervasive spore-forming bacteria and their spores increase proportionally. Recent reports demonstrate that spore-forming species in environmental biofilms form fruiting bodies that rise above the biofilm to facilitate the spore release [Branda et al., 2001, PNAS, 98, 11621-11626].

The present disclosure provides that bacterial endospore concentration is an indicator of microbial contamination in an indoor environment. In accordance with the disclosed method, a measurement of the bacterial endospore concentration of an indoor environment correlates to the microbial mass in said indoor environment.

The present disclosure further provides that the Airborne Endospore Bioburden (AEB) can be used as an indicator of spacecraft cleanliness. AEB, as measured in closed environment air sampling under laboratory conditions and in the Environmental Control and Life Support System at Marshall Space Flight Center, has indicated that increased total counts of airborne endospores can be correlated to surface microbial contamination.

The present disclosure provides that airborne bacterial spores are a good indicator of total biomass, because 1) airborne bacterial spores are easily quantified, and 2) airborne bacterial spore concentrations correlate to the total biomass in an indoor environment.

The method disclosed herein can be used to measure the concentration of airborne bacterial spores as an indicator of total microbial contamination of an environment. The method disclosed herein can be used to measure the concentration of airborne bacterial spores as an indicator of total microbial contamination of an indoor environment. The method disclosed herein can measure better than 0.1 bacterial spores per liter of air, which ensures accurate measurement of the background concentrations of bacterial spores in an indoor environment.

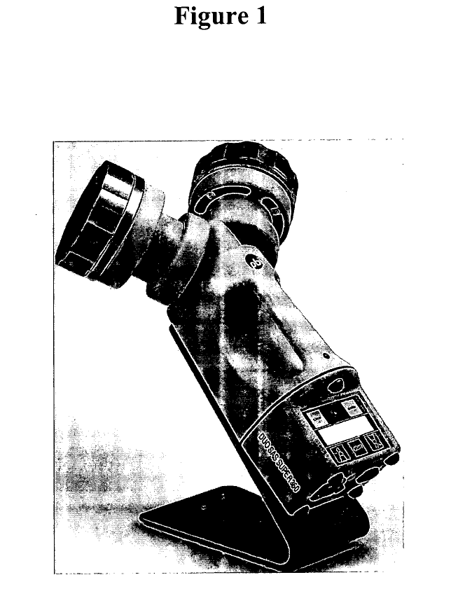

In one embodiment, the method by which the endospore concentration is measured is disclosed in U.S. patent application Ser. No. 11/332,788 incorporated herein by reference in its entirety. In order to measure the bacterial endospore concentration of the indoor environment using this method, the endospores are first captured from the air within the indoor environment. Bacterial spores in the air of the indoor environment are transferred from the air onto an air filter using an air sampler (FIG. 1)(Example 1).

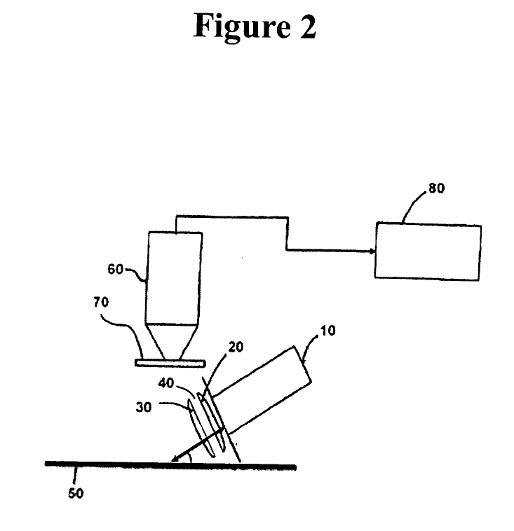

In another embodiment, after the bacterial endospores are captured (e.g. using an air sampler with an air filter), the endospores on the air filter are transferred to a test surface, onto which one or more lanthanide ions (terbium or europium) are provided. The DPA is then released from the spores onto the test surface either by lysing methods (autoclaving, microwaving, etc) or inducing germination (e.g. addition of L-alanine to the test surface). The released DPA binds with the lanthanide ions to form a complex on the test surface. This DPA-lanthanide ion complex is excited via a UV source (from a Xenon flash lamp, laser or UV LED), and the resulting luminescence is imaged and quantified using lifetime gated imaging (FIG. 2) Example 3)(Also see U.S patent application Ser. No. 11/332,788).

Thus, while a number of methods can be used to quantify airborne bacterial spore concentrations, lifetime gated imaging of bacterial spores coupled to an air sampler is a desirable method. The imaging method takes advantage of the fact that a bacterial spore contains 10⁸ molecules of DPA. The release of DPA by germination or lysis of the spore in the presence of a lanthanide ion results in a complex which upon UV activation forms a luminescent “halo” surrounding the spore body. Using a lifetime-gated imaging, this method can enumerate individual spores.

A microbial event as defined herein is a large change in microbial concentration over a defined period of time. In a closed-loop system such as a spacecraft cabin environment, microbial events can occur almost instantaneously—e.g. during failure in air filtration, or rupturing of wastewater vessel. Microbial events can also happen over weeks as biofilm formation increases, fostered by favorable temperatures, humidity, or nutrient availability.

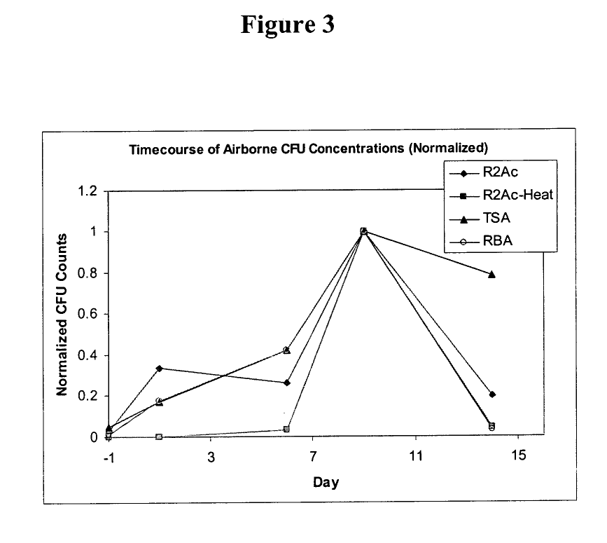

To demonstrate that the time course in an indoor environment of airborne bacterial spores shows a trend that is highly correlated to the time course of environmental, human commensal, and fungal spore concentrations, an experiment was carried out in a simulated spacecraft as detailed in Example 1. The results, as shown in FIG. 3, imply that a change in airborne bacterial spore concentration can be used to indicate a corresponding change in microbial content in an indoor environment such as a spacecraft cabin.

The shape of the time course in FIG. 3, across all media is very similar, including heat shock survivors (i.e., endospores), indicating a strong correlation between airborne spore increase and the overall increase of the bioburden. These results of sampling show that bacterial spore bioburden is an indicator of the microbial content of an environmental system. Time-course data indicates an increase in microbial counts during the time of occupation and a decrease after the environment is vacated.

The air sampler of the present disclosure can be of any type so long as the rate at which it samples the air (usually by way of a vacuum) is conducive to capturing spores from the air onto the surface with a high efficiency such as the SAS air sampler shown in FIG. 1.

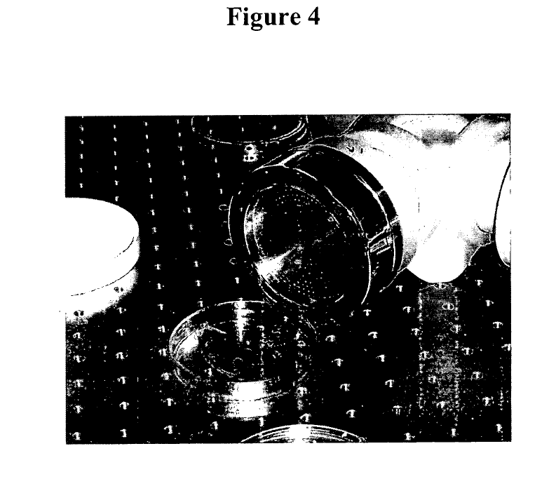

Bacterial endospores grown in surface biofilms on microbial growth media were shown to undergo airborne transport as a result of mild air turbulence under ambient conditions (FIG. 4)(Example 2). This occurrence indicates that a bacterial endospore concentration can be obtained from biofilms present in an indoor environment. This finding further demonstrates that under laboratory conditions, surface microbial biofilms can release bacterial endospores that can be transported through the air, resulting in the airborne spread of potentially dangerous microorganisms from surfaces.

The present disclosure is a rapid method for monitoring microbial concentrations in water, on surfaces and in the air. A system such as this can be referred to as a Microbial Event Monitor (MEM). Such a system, which is automated, continuously monitors the Airborne Endospore Bioburden (AEB) as an indicator of the microbial concentration in an indoor environment.

Such a system, in the least, comprises an air sampler, a spore lysis or germination system, a source of lanthanide ions, a spore-specific fluorescent indicator and a photo detector. From this chemical test, total and viable bacterial spore concentrations can be determined. The system is amenable to complete automation, and can be performed on the timescale of minutes.

The present application provides the ability to measure endospore concentration as an indicator of microbial contamination in realistic environments, as well as in a laboratory. The method provided herein allows for an increase in sensitivity by at least 2 logs as compared with colony assaying.

One embodiment of the present disclosure is a method that can detect single endospores. To replace colony culturing, which quantifies bacteria in colony forming units (CFUs) over the course of several days, a single spore assay according to U.S patent application Ser. No. 11/332,788, quantifies germinating spore units (GSU) in several minutes. Preferably the method is sensitive to 0.1 endospores/liter of air, and can obtain a bacterial endospore concentration for an indoor environment in less than 24 hours.

One of skill in the art can use alternative methods for obtaining the bacterial endospore concentration in an indoor environment. Any method for measuring bacterial endospore concentration serves as an indicator of microbial contamination.

For example, the indoor environment, could be swabbed, and the swabs subsequently analyzed as disclosed in U.S. patent application Ser. No. 11/332,788. Additionally, any liquid present in an indoor environment could be analyzed using the method disclosed in U.S. patent application Ser. No. 11/332,788.

### EXAMPLE 1

Culture-based Air Sampling in a Spacecraft Indoor Environment.

A Surface Air Systems (SAS) Super 360 dual head impaction air sampler was used to sample 1000 Liters of air on four media types in duplicate. Indoor air was sampled for 5 hours at 16.7 L/min (approximate sample rate that should collect the same size fraction and concentration of aeroallergens and aeropathogens as are inhaled and collected by the human respiratory system).

Samples were Collected on the Microbial Growth Media Listed Below:

RBA (Rose Bengal Agar) for enhanced fungal spore growth (incubated at room temperature—RT).
TSA (Tryptic Soy Agar) for enriched growth of microorganism (incubated at 35° C.).
R2A for environmental microorganisms (incubated at RT).
R2A (heat shock at 85° C.) for environmental bacterial spore-formers (incubated at RT).

Sampling began one day prior to occupation, with chamber sealed for 24 hours to allow for environmental equilibration. Sampling continued on days 1, 6, 9 and 14 of occupation. All sampling was conducted at the rear right section of the cabin. FIG. 3 shows the time course data for airborne microbial concentrations collected onto the four media types.

Microbial Isolates Included:
R2A_{C}—B. pumilus, B. megaterium, Coagulase-negative Staphylococcus, Micrococcus, Corynebacterium 1 & 2, Non-fermentable, gram negative rod.
R2A_{C}-Heat—B. pumilus, Non-fermentable, gram negative, rod, Corynebacterium.
TSA—B. pumilus, B. cereus, Coagulase-negative Staphylococcus, Micrococcus, Corynebacterium 1 & 2.
RBA—Penicillium, Cladosporium 1 & 2, Non-sporulating hyphae, yeast, Epicoccum, Nigrospora, Curvularia.

### EXAMPLE 2

Aerosolized Biofilm Endospore Testing in the Laboratory

Bacterial endospores grown in surface biofilms on microbial growth media were shown to undergo airborne transport as a result of mild air turbulence under ambient conditions. Cultures of bacterial endospores (Bacillus subtilis (BS)-3160) were grown on Tryptic Soy Agar (TSA) bacterial growth medium. Two weeks after the culture was initiated, to allow time for the formation of bacterial endospores on the surface of the biofilm, an SAS air sampler was used to measure the concentration of spores which could easily be transported by moderate air flow over the surface of the biofilm.

### EXAMPLE 3

Detection and Quantifying Apparatus

An apparatus for detecting and quantifying bacterial spores on a surface including lanthanide ions and aromatic molecules released from the bacterial spores on the surface. The apparatus in FIG. 2 comprises a UV-light radiation device (10) for exciting a complex of a Tb³⁺ion and DPA/DP to generate a characteristic luminescence of the complex on a surface. The source for the UV-light was a Xenon flash lamp, which was approximately 5 cm away the test surface (50). Between the Xenon flash lamp and the test surface were two C-amount elliptical lenses (20, 30) (40 represents the space in between the lenses). The Xenon flash lamp and the test substrate were positioned at an angle of 45 degrees to each other. The area of irradiation by the Xenon flash lamp was observed by a microscope objective (60) with a red bandpass filter (70) suitable for detecting and quantifying bacterial spores exhibiting the luminescence of the complex on the surface. The image was transferred from the microscope to the imaging device (80) for imaging bacterial spores exhibiting the luminescence, using an imageX nanoCCD camera (Photonic Research Systems Ltd, United Kingdom). The pixel size on the camera is 11.6 microns horizontal by 11.2 microns vertical and the camera has a chip with 752×582 pixels on a 10.25 mm×8.5 mm vertical area. Lifetime gated images were captured with a 100-μs delay integrating for 2 milliseconds. 6 to 13 images were taken over different areas of the medium. Each image captured an actual agarose area of 3.2 mm² at 40× magnification. The spatial resolution is a function of the camera, the camera objective and the microscope camera port. The microscope image is projected onto the camera port that then determines the spatial resolution.

While several illustrative embodiments have been shown and described in the above description, numerous variations and alternative embodiments will occur to those skilled in the art. Such variations and alternative embodiments are contemplated, and can be made without departing from the scope of the invention as defined in the appended claims.

1. An indicator of microbial contamination in an indoor environment, said indicator comprising measured bacterial endospore concentration in said indoor environment.

2. A method of using bacterial endospore concentration, comprising:
measuring said bacterial endospore concentration in an indoor environment;
correlating said measured bacterial endospore concentration to biomass in said indoor environment, thereby providing an indicator for said biomass.

3. A method for indicating presence of microbial contamination in an indoor environment, comprising measuring bacterial endospore concentration in said indoor environment to provide an indicator of said microbial contamination.

4. The method of claim 3 wherein the measuring comprises
capturing the bacterial endospores;
transferring the bacterial endospores to a test surface;
providing one or more lanthanide ions on the test surface;
releasing DPA from the bacterial spores on the test surface;
forming a complex of the one or more lanthanide ions and the DPA on the test surface;
exciting the complex to generate a characteristic luminescence of the complex on the test surface; and
imaging and quantifying the bacterial spores represented by the luminescence of the complex on the test surface.

5. The method of claim 4 wherein the capturing of the bacterial endospores is carried out using an air sampler.

6. The method of claim 4 wherein the imaging of the bacterial endospores is carried out using a lifetime-gated imaging.

7. The method of claim 4, wherein the one or more lanthanide ions comprises terbium ions.

8. The method of claim 4, wherein the one or more lanthanide ions comprises europium ions.

9. The method of claim 4, wherein exciting the complex is carried out using a UV source.
